(19) United States
(12) Patent Application Publication
(10) Pub. No.: US 2023/0381479 A1
(43) Pub. Date: Nov. 30, 2023

(54) SQEEZABLE SUBCUTANEOUS PORT

(71) Applicant: Portal Access, Inc., Wilmington (DE)

(72) Inventors: Michael Gabriel TAL, Tel Aviv (IL); Ron LIVNE, Haifa (IL); Omer EILON, Nachshonim (Kibbutz) (IL); Ronny Winshtein, Ramat HaSharon (IL)

(21) Appl. No.: 18/265,644

(22) PCT Filed: Dec. 8, 2021
(86) PCT No.: PCT/US2021/062403

§ 371 Date: Jun. 6, 2023

Related U.S. Application Data
(60) Provisional application No. 63/123,028, filed on Dec. 9, 2020.

(51) Int. Cl. A61M 39/02

(52) U.S. Cl. CPC A61M 39/0208; A61M 2039/0232; A61M 2205/0216

(57) ABSTRACT

Disclosed are a subcutaneous port and method of implantation thereof. The subcutaneous port comprising a rigid inner member and an outer member comprising of flexible material connected to the inner member along at least one lateral periphery portion of the inner member, thereby forming a predetermined spatial shape of the subcutaneous port when in an elastically relaxed state. The subcutaneous port is configured to squeeze into a subcutaneous void when pushed through a surgical opening greater than a maximal cross-sectional circumference of the inner member and smaller than a maximal cross-sectional circumference of the predetermined spatial shape.

### CROSS-REFERENCE TO RELATED APPLICATIONS

This application claims the benefit of U.S. Provisional Patent Application No. 63/123,028, filed on Dec. 9, 2020, titled “Squeezable Subcutaneous Port”, the entire contents of which are hereby incorporated by reference herein.

### FIELD OF THE INVENTION

The present disclosure relates to devices and methods for facilitating and/or improving repeated deliveries of fluids (e.g., fluids carrying nutrients, medicament and/or agents such as chemotherapy agents) into vasculature of a subject, and more particularly, but not exclusively, to vascular access ports and methods of delivery and deployment thereof in a body of a subject.

### BACKGROUND OF THE INVENTION

Repeated needle pricking for facilitating delivery or withdrawal of fluids (e.g., medication or agents) to patient's vascular system causes harm to local tissues and decreases target blood vessel functionality and needle placement accuracy. This phenomenon is often evident in chronic diabetes, dialysis or chemotherapy patients, for example, who require continuous and repeated intravenous fluids administration for prolonged periods.

A vascular access port is a device that enables such repeated pricking and fluid administration while minimizing the accumulated harm caused by needle pricking and powered injections of fluid. The access port is subcutaneously implanted, in a surgically formed pocket in proximity to a large blood vessel, usually in the chest. It is basically formed of a port body enclosing a cavity, which is capped with a septum member configured for supporting the upper skin layers and for accepting repeated needle pricking therethrough for intravascular fluid deliveries sealed to the surrounding body tissues. The port is attached to a catheter (a thin, flexible tube) which provides fluid communication with a large blood vessel, such as the superior vena cava, in order to allow the injected fluid to dilute in the blood stream.

The implantation of a port is considered a minor procedure performed under local or general anesthesia by an interventional radiologist or a surgeon. First, the surgeon achieve access to the desired vein, a skin incision is made afterwards in the access point. Second larger incision is made above the desired location of the port, through which a pocket-like subcutaneous void is made using blunt device. The catheter is extended subcutaneously between the two incisions using a blunt tunneler. One end of the catheter is then inserted into the vein and its other end is coupled to the port. Optionally, during deployment the catheter is cut to a desired length.

Besides progress made in past years in access ports design, there is still a need to develop ports and methods of implantation and deployment thereof, which are less traumatic and invasive, and simpler to perform, potentially also by non-surgical medical personnel.

### SUMMARY OF THE INVENTION

The present disclosure relates to devices and methods for facilitating and/or improving repeated deliveries of fluids (e.g., fluids carrying nutrients, medicament and/or agents such as chemotherapy agents) into vasculature of a subject, and more particularly, but not exclusively, to vascular access ports and methods of delivery and deployment thereof in a body of a subject.

In certain embodiments, there is provided a subcutaneous port. The subcutaneous port can comprise: a rigid inner member comprising a cavity opened to a first cavity opening closed with a septum member, configured for repeated needle penetrations therethrough into the cavity, and to a second cavity opening configured for facilitating fluid communication between the cavity and a lumen of a catheter; and an outer member comprising of flexible material connected to the inner member along at least one lateral periphery portion of the inner member thereby forming a predetermined spatial shape of the subcutaneous port when in an elastically relaxed state. In some embodiments, the subcutaneous port is configured to squeeze into a subcutaneous void when pushed through a surgical opening greater than a maximal cross-sectional circumference of the inner member and smaller than a maximal cross-sectional circumference of the predetermined spatial shape.

In some embodiments, the subcutaneous port in the elastically relaxed state is greater than the inner member by at least 50% in width, in area and/or in volume, in a maximal axial cross section of the predetermined spatial shape.

In some embodiments, the outer member is locally elastically compressible laterally towards the at least one lateral periphery portion of the inner member.

In some embodiments, the subcutaneous port is configured to reduce in maximal width by at least 10% when the outer member is compressed under a force greater than 5 N, and/or by at least 25% when the outer member is compressed under a force greater than 20 N.

In some embodiments, the outer member is configured to conform to a locally radially compressed shape while radially expanding remotely to a compressed region thereof.

In some embodiments, the outer member is extendable proximally relative to the inner member into an extended shape narrower and longer than the predetermined spatial shape.

In some embodiments, the outer member is configured with elastic resistance to compression sufficient to maintain the predetermined spatial shape when under naturally occurring subcutaneous stresses in the subcutaneous void.

In some embodiments, the subcutaneous port substantially maintains volume thereof, when the outer member is compressed or drawn proximally relative to the inner member from the predetermined spatial shape.

In some embodiments, the flexible material includes soft elastomer and/or silicone rubber.

In some embodiments, the flexible material fills most or all space formed in the predetermined spatial shape around the inner member.

In some embodiments, the subcutaneous port comprising at least one elastic extension stiffer than the flexible mated al, projecting from the inner member and surrounding the at least one lateral periphery portion of the inner member, wherein the at least one extension is embedded in the flexible material and configured to distribute compressing loads originating from a locally compressed portion to other portions of the outer member.

In some embodiments, the at least one extension forms a gap with the at least one lateral periphery portion of the inner member filled with the flexible material.

In some embodiments, the at least one extension is configured to approximate the at least one lateral periphery portion of the inner member when the outer member is compressed laterally and/or extended axially proximally relative to the inner member.

In some embodiments, the at least one extension projects proximally and laterally-outwardly from a distal portion of the inner member located distally to the cavity.

In some embodiments, the at least one extension is fixed to the inner member distal portion and allowed to flex axially and/or laterally relatively to the inner member with portions thereof distant to the inner member distal portion.

In some embodiments, the at least one extension encircles or surrounds most or all lateral periphery of the inner portion.

In some embodiments, the outer member is locally compressible against the inner member to about 50% or less its elastically unstressed width, adjacent to the maximal cross-sectional circumference of the predetermined spatial shape, when forced through the surgical opening.

In some embodiments, the outer member is locally compressible against the inner member to about 25% or less its elastically unstressed width, adjacent to the maximal cross-sectional circumference of the predetermined spatial shape, when forced through the surgical opening.

In some embodiments, the outer member is locally compressible against the inner member sufficiently for squeezing through the surgical opening when the subcutaneous port is pushed with an axial force equal to or smaller than about 5 kgf.

In some embodiments, the flexible material includes elastic polymer configured with hardness equal to or smaller than about 35 Shore A.

In some embodiments, wherein the inner member extends longitudinally along most or all length of the subcutaneous port.

In some embodiments, the inner member includes a distal portion extending distally relative to the cavity, the distal portion having a rounded or pointed leading edge and/or is configured to facilitate or ease penetration of the subcutaneous port via the surgical opening.

In some embodiments, the outer member varies in width and/or thickness in at least one direction relative to the inner member.

In some embodiments, the inner member includes a distal portion, an intermediate portion and a proximal portion, wherein the outer member is greater in width and/or thickness along the intermediate portion and/or along the proximal portion than along the distal portion.

In some embodiments, the inner member includes a superior portion and an inferior portion, wherein the outer member is greater in width and/or thickness along the inferior portion than along the superior portion.

In some embodiments, the outer member lengthens when is locally compressed against the inner member, thereby increasing overall length of subcutaneous port.

In some embodiments, the outer member is configured to lengthen mostly or only proximally, and/or the inner member is configured to resist or prevent the outer member from lengthening distally, relative to the inner member.

In some embodiments, the outer member expands inferiorly to a base of the inner member, perpendicularly to the compression direction, when locally compressed against the inner member, thereby increasing overall height of the subcutaneous port.

In some embodiments, the outer member is configured to flex or expand mostly or only inferiorly, and/or the inner member is configured to resist or prevent the outer member from expanding superiorly.

In some embodiments, a minimal force sufficient to flex or expand the outer member inferiorly, below the base of the inner member, is smaller than a minimal force sufficient to flex or expand the outer member superiorly.

In some embodiments, the septum member is oval.

In some embodiments, the subcutaneous port comprising a rigid grasping portion at proximal end thereof configured for facilitating grasping with grasping means, such as Kelly clamps or surgical needle holder.

In some embodiments, the rigid grasping portion includes a flat surface extending horizontally so as to restrict a surgical needle holder grasping the rigid grasping portion when arms thereof are arranged vertically, relative to the subcutaneous port.

In some embodiments, the subcutaneous port comprising a cap member coupled over the septum member to a superior portion of the inner member to form a unitary rigid encapsulated core body of the subcutaneous port.

In some embodiments, the encapsulated core body is configured to withstand power injection pressures generatable within the cavity.

In some embodiments, the outer member is formed as a single component.

In some embodiments, the outer member is formed by way of extruding, casting or molding the flexible material over the inner member within restricting spatial boundaries shaped according to the predetermined spatial shape.

In some embodiments, the outer member is extruded, casted or molded over the encapsulated core body to form the subcutaneous port.

In some embodiments, the outer member is formed as a solid member occupying substantially most or all volume thereof around the inner member.

In some embodiments, the outer member includes an elastic shell-like structure comprising a thin layer enclosing an at least one outer member cavity in each side of the inner member.

In some embodiments, the at least one outer member cavity is at least partly filled with the flexible material.

In some embodiments, the shell-like structure is formed of a first material and the flexible material is formed of a second material, wherein the first material differs from the second material by at least one of rigidity, elasticity and compression strength.

In some embodiments, hardness of the first material is about 30 Shore A to about 50 Shore A, and hardness of the second material is about 00-20 Shore to about 20 Shore A.

In some embodiments, the outer member includes a plurality of thru holes having aggregated empty volume occupying at least 20% of the outer member total volume.

In some embodiments, the outer member includes a plurality of thin rib-like members each extending perpendicularly from side of the inner member, the rib-like members are spaced apart from each other sufficiently to allow a first phase of partial flexing of the rib-like members without contacting each other, when the subcutaneous port is pushed through the surgical opening.

In some embodiments, the outer member has a substantially oval shaped base footprint.

In some embodiments, the outer member has a base footprint shape formed of sequentially joined oval segments forming narrowed neck portions therebetween.

In certain embodiments there is provided a method for implanting a subcutaneous port. The method can comprise at least one of the following steps (not necessarily in same order):
forming a surgical opening across skin layers of a subject, the surgical opening comprising an opening neck portion enclosing and restricting a maximal opening circumference;
creating a subcutaneous void beneath the skin layers via the surgical opening;
pushing the subcutaneous port into the subcutaneous void via the surgical opening, the subcutaneous port has a predetermined spatial shape having a maximal cross-sectional circumference greater than the maximal opening circumference when in an elastically relaxed state, the subcutaneous port is locally elastically compressible along a length thereof.

In some embodiments, the pushing forces the subcutaneous port to elastically compress in diameter and/or extend proximally in length when pressed against the opening neck portion, thereby allowing squeezing of the subcutaneous port through the surgical opening.

In some embodiments, following the pushing, the method comprising allowing the subcutaneous port to voluntarily expand elastically up to the elastically relaxed state.

In some embodiments, the creating and the pushing is performed with a surgical instrument:

In some embodiments, the surgical instrument is a Kelly clamps or a surgical needle holder.

In some embodiments, the subcutaneous port includes a flexible outer member connected to a rigid inner member along at least one lateral periphery portion of the inner member thereby forming the predetermined spatial shape of the subcutaneous port when in an elastically relaxed state.

In some embodiments, the rigid inner member comprising a cavity opened to a first cavity opening closed with a septum member, configured for repeated needle penetrations therethrough into the cavity, and to a second cavity opening configured for facilitating fluid communication between the cavity and a lumen of a catheter.

In some embodiments, the outer member is configured with elastic resistance to compression sufficient to maintain the predetermined spatial shape within a surgically formable subcutaneous void when under naturally occurring subcutaneous stresses.

In some embodiments, the pushing forces the outer member to compress locally against the inner member while substantially maintaining an overall volume thereof by enlarging remotely to a compressed region thereof.

In some embodiments, the method further comprising at least one of: accessing into the jugular vein with a needle, inserting a wire into the jugular vein through the needle, removing the needle from the jugular vein, inserting a peel apart sheath and/or a dilator into the jugular vein over the wire, removing the wire and/or the dilator from the jugular vein, inserting a catheter into the jugular vein through the peel apart sheath, and removing the peel apart sheath from the jugular vein.

In some embodiments, the method further comprising at least one of: advancing the catheter via the jugular vein to the superior vena cava, confirming under imaging position of a distal tip of the catheter in the superior vena cava or in the right atrium, and adjusting the position of the catheter distal tip.

In certain embodiments there is provided a method for removing a subcutaneous port from a body of a subject. In some embodiments, the method comprising at least one of the following steps (not necessarily in same order):
forming a surgical opening across skin layers of the subject, the surgical opening comprising an opening neck portion enclosing and restricting a maximal opening circumference;
creating a subcutaneous passage beneath the skin layers between the surgical opening and a proximal end of the subcutaneous port;
pulling the subcutaneous port through the subcutaneous passage and the surgical opening for removing it from the patient.

In some embodiments, the subcutaneous port has a predetermined spatial shape having a maximal cross-sectional circumference greater than the maximal opening circumference when in an elastically relaxed state, the subcutaneous port is locally elastically compressible along a length thereof.

In some embodiments, the pulling forces the subcutaneous port to elastically compress in diameter and/or extend proximally in length when pressed against the opening neck portion, thereby allowing squeezing of the subcutaneous port through the surgical opening.

In some embodiments, the creating and the pulling is performed with a surgical instrument.

In some embodiments, the surgical instrument is a Kelly clamps or a surgical needle holder.

In some embodiments, the subcutaneous port includes a flexible outer member connected to a rigid inner member along at least one lateral periphery portion of the inner member thereby forming a chosen predetermined spatial shape of the subcutaneous port when in an elastically relaxed state.

In some embodiments, the rigid inner member comprising a cavity opened to a first cavity opening closed with a septum member, configured for repeated needle penetrations therethrough into the cavity, and to a second cavity opening configured for facilitating fluid communication between the cavity and a lumen of a catheter.

In some embodiments, the outer member is configured with elastic resistance to compression sufficient to maintain the chosen predetermined spatial shape within the subcutaneous passage.

In some embodiments, the pulling forces the outer member to compress locally against the inner member while substantially maintaining an overall volume thereof by enlarging remotely to a compressed region thereof.

In some embodiments, the surgical opening is formed adjacent to or over an insertion scar previously made for implanting the subcutaneous port.

All technical or/and scientific words, terms, or/and phrases, used herein have the same or similar meaning as commonly understood by one of ordinary skill in the art to which the invention pertains, unless otherwise specifically defined stated herein. Illustrative embodiments of methods (steps, procedures apparatuses (devices, systems, components thereof), equipment, and materials, illustratively described herein are exemplary and illustrative only and are not intended to be necessarily limiting. Although methods, apparatuses, equipment, and materials, equivalent or similar to those described herein can be used in practicing or/and testing embodiments of the invention, exemplary methods, apparatuses, equipment, and materials, are illustratively described below. In case of conflict; the patent specification, including definitions, will control.

### BRIEF DESCRIPTION OF THE DRAWINGS

Some embodiments are herein described, by way of example only, with reference to the accompanying drawings. With specific reference now to the drawings in detail; it is stressed that the particulars shown are by way of example and for purposes of illustrative description of some embodiments. In this regard, the description taken together with the accompanying drawings make apparent to those skilled in the art how some embodiments may be practiced.

In the drawings:

FIGS. 1A-1C schematically illustrate respectively a side cross-sectional view and a top cross-sectional view of an exemplary deployed vascular access port, in accordance with some embodiments;

FIGS. 2A-2F schematically illustrate exemplary scenarios representing steps in an exemplary procedure for implanting the exemplary squeezable subcutaneous port shown in FIG. 1A, according to some embodiments;

FIGS. 3A-3C schematically illustrate exemplary scenarios representing steps in an exemplary procedure for accessing and operating the exemplary squeezable subcutaneous port shown in FIG. 1A, according to some embodiments;

FIGS. 3D-3E schematically illustrate exemplary scenarios representing steps in an exemplary procedure for accessing an exemplary variation of the squeezable subcutaneous port shown in FIG. 1A, according to some embodiments;

FIGS. 4A-4B respectively illustrate an exemplary squeezable subcutaneous port in an assembled isometric view and in an exploded isometric view, according to some embodiments;

FIGS. 5A-5B respectively illustrate the exemplary squeezable subcutaneous port shown in FIG. 4A in a side cross-sectional view and in a frontal cross-sectional view, according to some embodiments;

FIG. 6 illustrates the exemplary squeezable subcutaneous port shown in FIG. 4A grasped with an exemplary surgical needle holder, according to some embodiments;

FIG. 7A illustrates an exemplary squeezable subcutaneous port with an outer member shown in FIGS. 7B and 7C, which includes empty elastic shell-like structure, according to some embodiments;

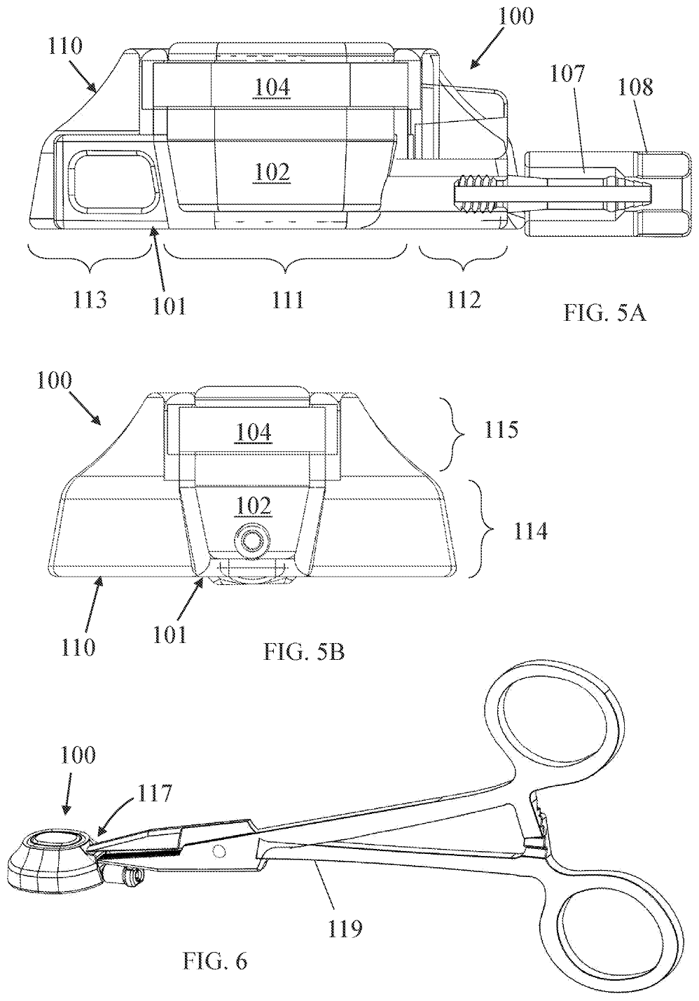

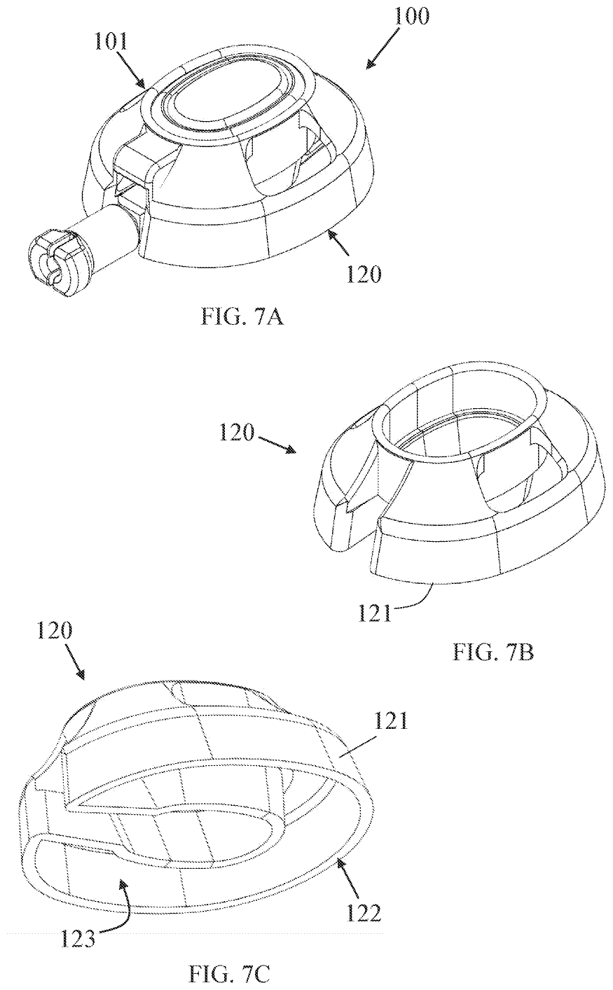

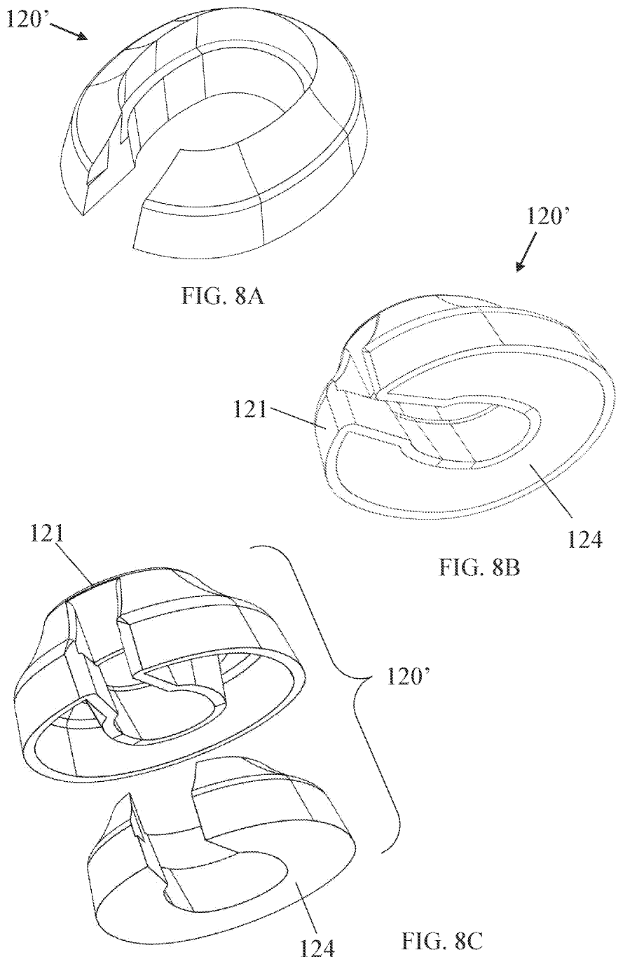

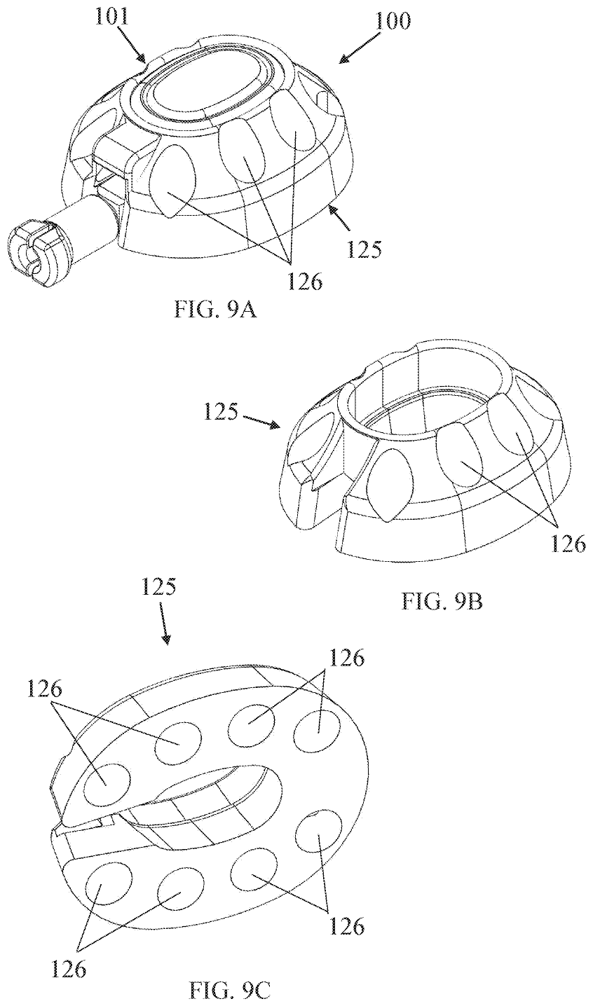

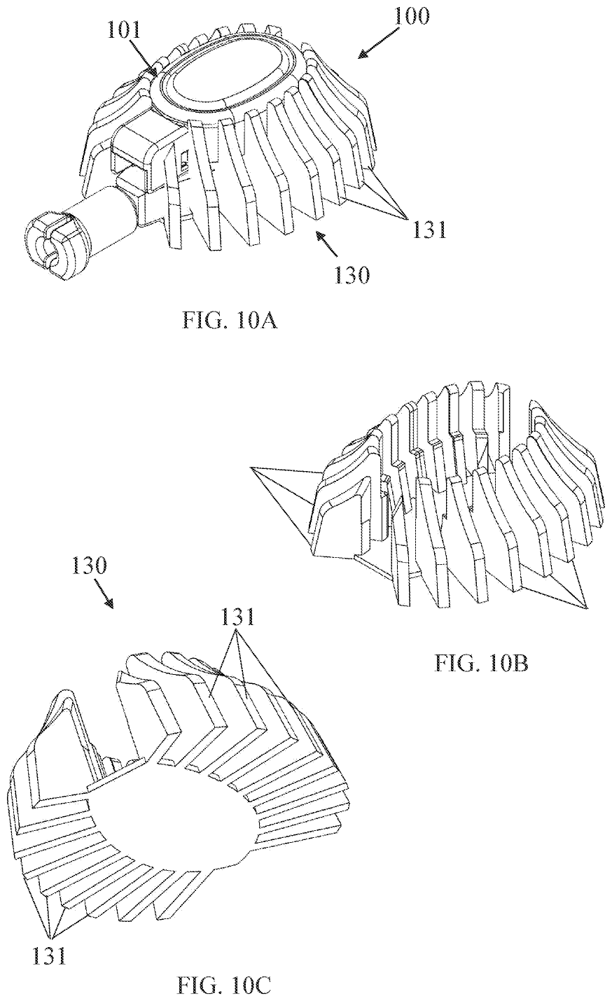

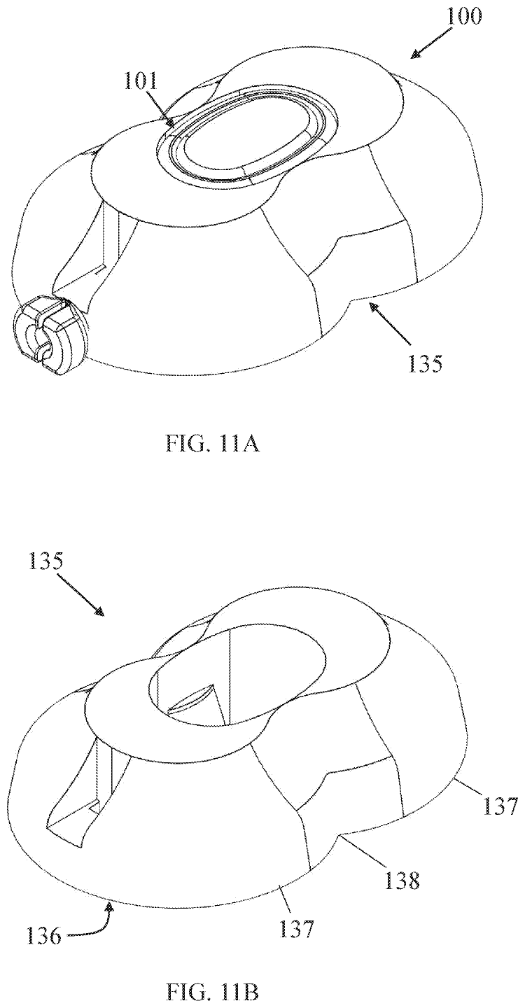

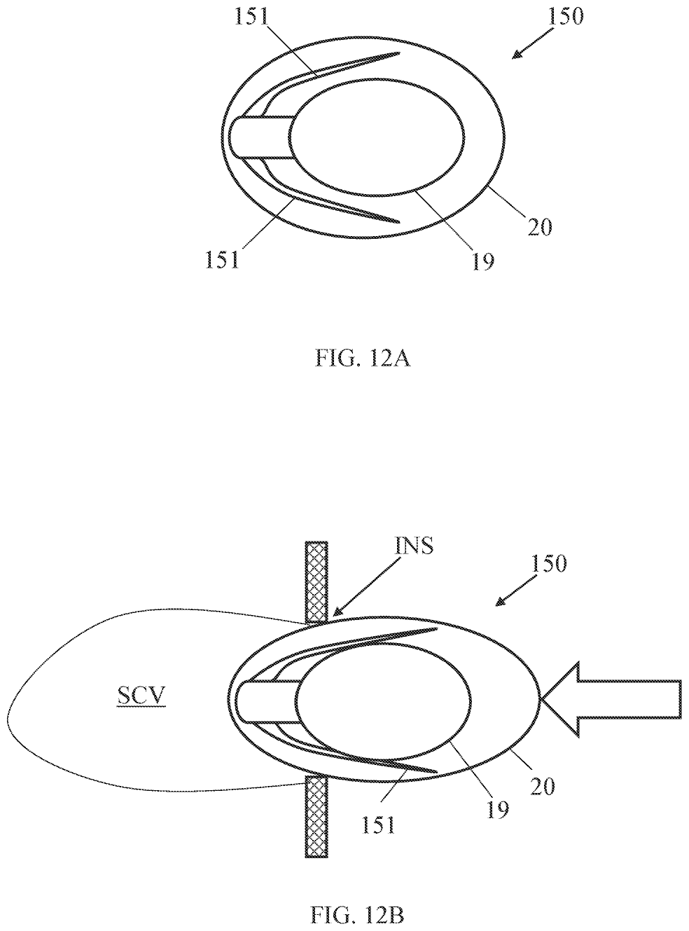

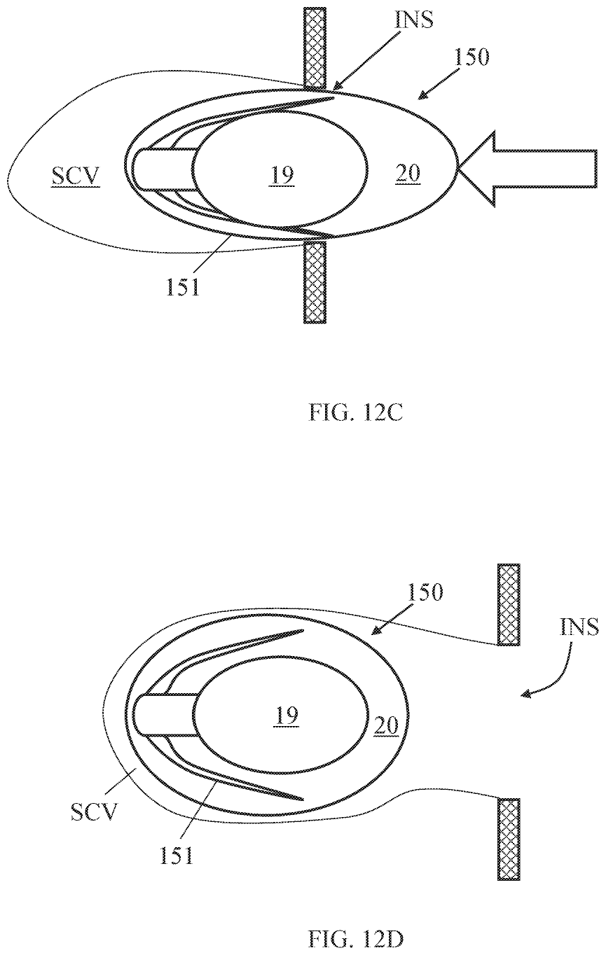

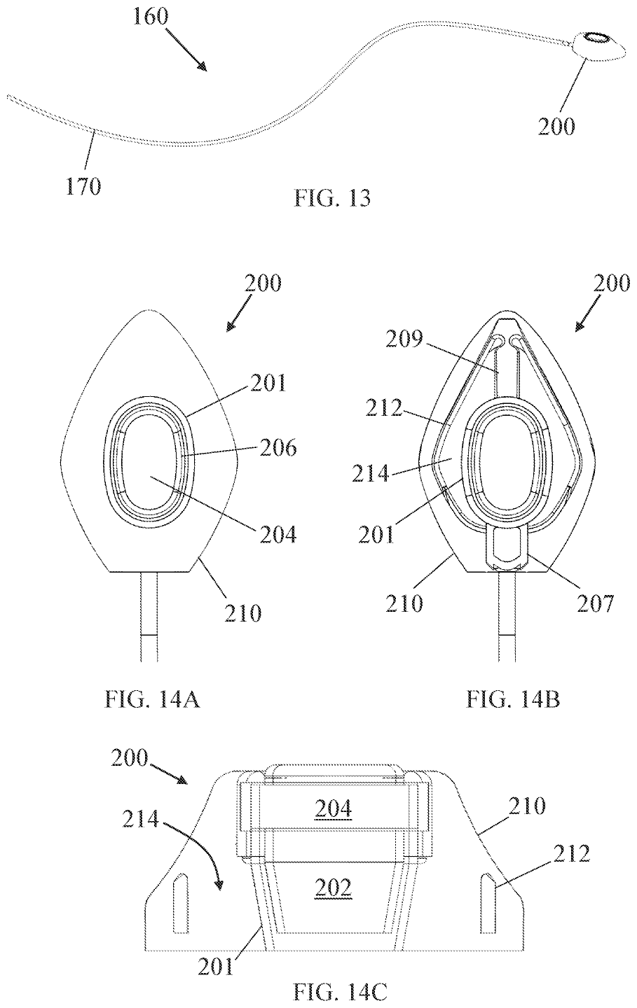

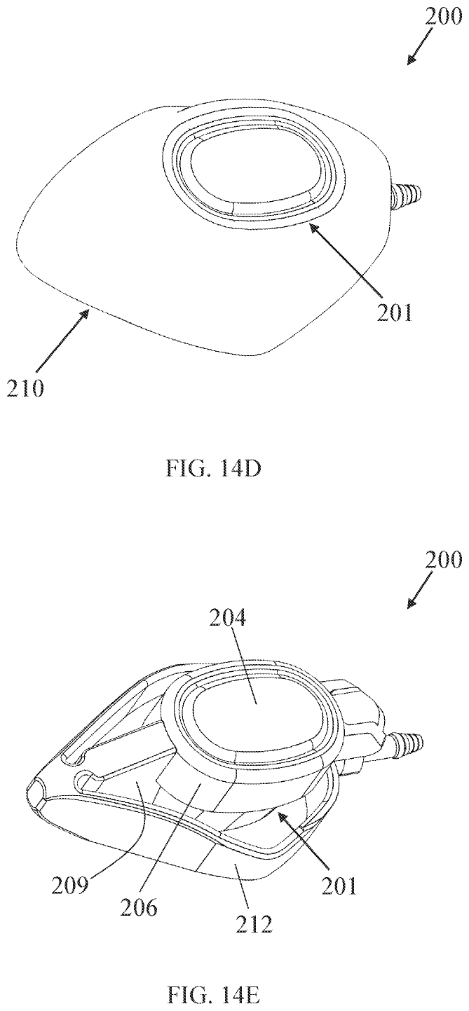

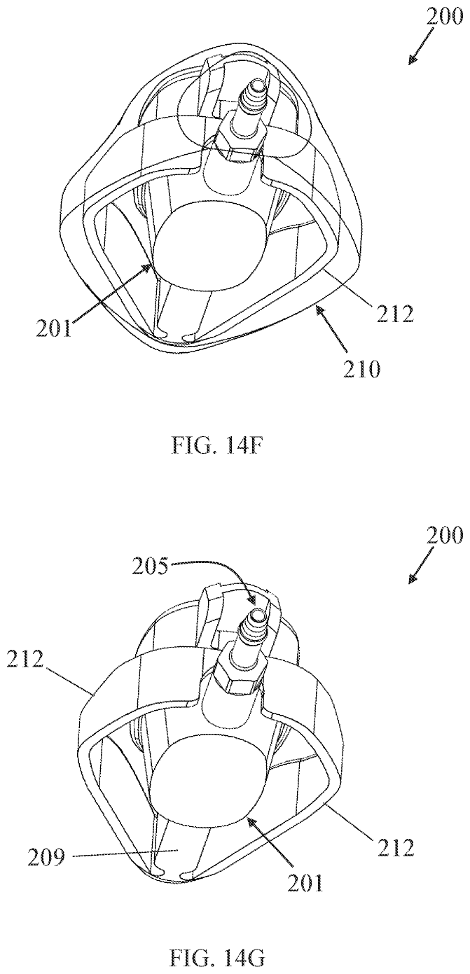

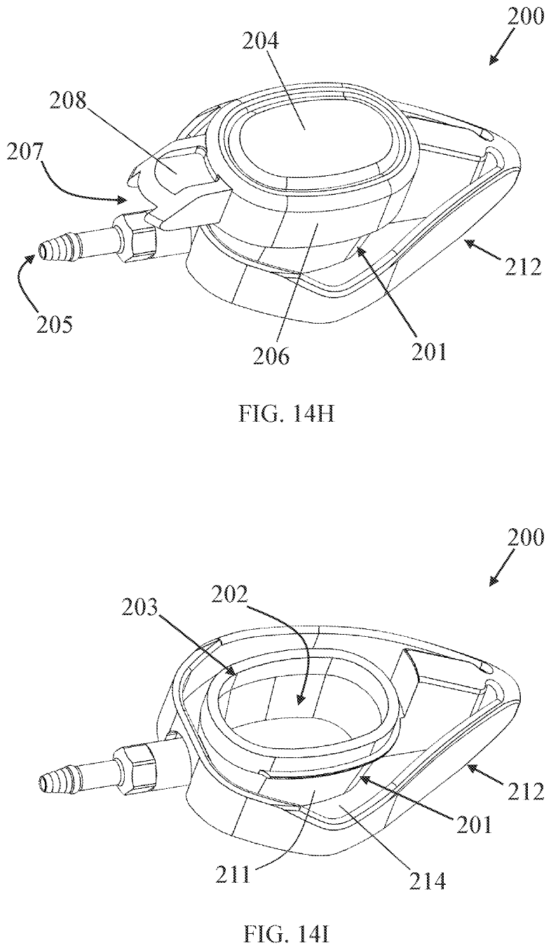

FIGS. 5A-8C illustrate an exemplary variant of the outer member shown in FIG. 7B, which includes elastic shell-like structure filled with flexible filler, according to some embodiments;

FIG. 9A illustrates an exemplary squeezable subcutaneous port with an outer member shown in Ms. 9B and 9C, which includes a plurality of thru holes, according to some embodiments;

FIG. 10A illustrates an exemplary squeezable subcutaneous port with an outer member shown in FIGS. 10B and IOC, which includes a plurality of thin rib-like members, according to some embodiments;

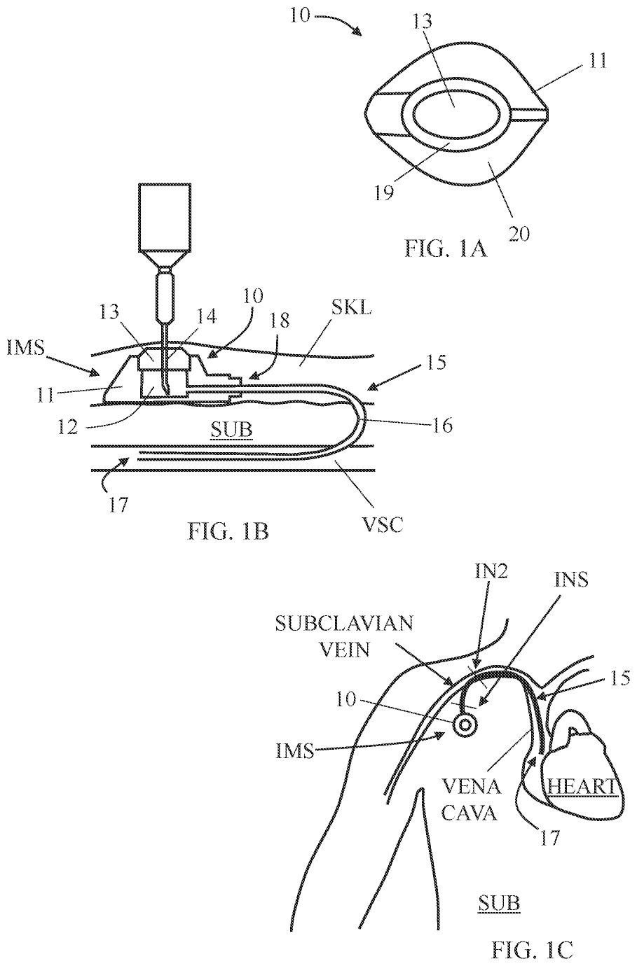

FIG. 11A illustrates an exemplary squeezable subcutaneous port with an outer member shown in FIG. 1.1B having a base footprint shape formed of sequentially joined oval segments, according to some embodiments;

FIGS. 12A-12D schematically illustrate exemplary scenarios representing steps in an exemplary procedure for implanting an exemplary squeezable subcutaneous port comprising an elastic extension embedded in a flexible material, according to some embodiments;

FIG. 13 illustrates an exemplary vascular access port system comprising an exemplary subcutaneous port connected to a catheter, according to some embodiments; and

FIGS. 14A-14I illustrate several views of the subcutaneous port shown in FIG. 13, according to some embodiments.

### DETAILED DESCRIPTION

The present disclosure, in some embodiments thereof, relates to devices and methods for facilitating and/or improving repeated deliveries of fluids (e.g., fluids carrying nutrients, medicament and/or agents such as chemotherapy agents) into vasculature of a subject, and more particularly, but not exclusively, to vascular access ports and methods of delivery and deployment thereof in a body of a subject. In some embodiments, vascular access ports of the present disclosure can improve safety and/or efficacy of the surgical implantation procedure of access port and catheter by reducing size or number of surgical processes (like cuts, incisions, and tunneling), their duration and/or complexity, thereby offering a potentially less traumatic experience with shorter, easier recovery for the patient.

FIGS. 1A-1C schematically illustrate respectively a side cross-sectional view and a top cross-sectional view of an exemplary vascular access port 10, optionally configured as a squeezable subcutaneous port capable of penetrating through a small opening, such as one formed by puncture or incision made to patient's skin, incapable of accommodating passage therethrough of port 10 in its maximal cross sectional circumference when in an elastically relaxed state. Penetration through such an opening can be accomplished by forcing one or more portions of port 10 to elastically compress locally by the opening neck portion, when it is pushed distally through the opening. As used herein, the term “cross sectional circumference” refers to perimeter or arc length in a particular cross section of an object such as subcutaneous port (in this embodiments, port 10), or part thereof, crossing transversely an axis, such as a long or longitudinal axis, that extends between distal and proximal ends of the object (e.g., subcutaneous port). The maximal cross-sectional circumference means the cross-sectional circumference having greatest length among all cross-sectional circumferences.

Vascular access port 10, shown in top view in FIG. 1A and inside cut view in FIG. 1B, includes a port body 11 defining a cavity 12 and coupled with a septum member 13 that covers and seals cavity 12 from surroundings. Septum member 13 is configured for repeated puncturing of needles, like needle 14 shown in FIG. 1B, without compromising sealing of cavity 12 during needle placement therethrough and after the needle is withdrawn. “Repeated” in this context may refer to more than 10 consecutive needle punctures, optionally more than 100 consecutive needle punctures, optionally more than 1,000 consecutive needle punctures, optionally more than 10,000 consecutive needle punctures, or higher or lower. “Needle” in this context may refer to needles approved for fluid deliveries through vascular access ports, such as for intravenous administration.

Port 10, and particularly port body 11, includes a rigid inner member 19, which forms cavity 12, and a flexible outer member 20 (formed of a flexible material) connected to inner member 19 along at least one lateral periphery portion of the inner member, and forming a chosen predetermined spatial shape for port 10 (as shown in FIG. 1A, for example) when in an elastically relaxed state. Outer member 12 is configured with elastic resistance to compression sufficient to maintain the predetermined spatial shape within a surgically formable subcutaneous void when under naturally occurring subcutaneous stresses. Furthermore, outer member 20 is locally compressible against inner member 19, and configured to substantially maintain an overall volume by enlarging remotely to a compressed region thereof, thereby facilitating squeezing of port 10 into the subcutaneous void when pushed through a surgical opening greater than a maximal cross sectional circumference of the inner member and smaller than a maximal cross sectional circumference of the predetermined spatial shape.

Port 10 is implantable in a target implantation site IMS subcutaneously beneath skin layers SKI (including optionally within or beneath fat tissue) in a subject SUB. When fully deployed, vascular access port 10 has cavity 12 in fluid communication with vasculature VSC of subject SUB, normally a large blood vessel such as the Subclavian vein or the Vena Cava, so that fluid administrated into cavity 12 via needle 14 will flow directly to the subject's vascular system. A catheter 15 with catheter lumen 16 has a first catheter end 17 thereof positioned in and opened to vasculature VSC, and a second catheter end 18 thereof is connected to port body 11 and opened to cavity 12; catheter ends, 17 and IS, are opened to catheter lumen 16 and facilitate fluid communication between cavity 12 and vasculature VSC. FIG. 1B shows an optional deployment scheme where port 10 is positioned on an upper part the subject's chest in proximity to access opening made to Jugular vein, with first catheter end 17 positioned in the Vena Cava in proximity to subject's right atrium. Vascular access port 10 may be provided separately to catheter 15 with a connector configured for selective connection therebetween, optionally within the body, or alternatively vascular access port 10 and catheter 15 are provided together as an assembly kit or as a unified device.

As used herein, the term “vascular access port” refers to an implant intended for repeated transfer of fluids administered to and/or withdrawn from a subject. The disclosures described herein are advantageous also when used in conjunction with vascular access ports that have a septum member configured for repeated puncturing by a needle, but this particular feature is not a requirement and other forms of needle access openings or platforms may apply. Some vascular access ports described herein include one or more components configured, collectively, when properly assembled and deployed, for prolonged implantation in a live (e.g., humans subject and for repeated fluid transfer access, such as through a septum member. The vascular access port includes at least a structural object referred to herein as a “port body” which serves as a facilitating structure for fluid transfer access and/or as a support structure configured for holding components (e.g., a septum) applicable for fluid transfer access.

In some embodiments, the port body forms a cavity beneath (e.g., inferiorly to) the needle access opening or septum, which is sized and shaped for repeatedly receiving a needle tip, for accumulating a chosen or predetermined volume of fluid (e.g., a liquid such as a solution, a suspension or a colloid), and/or for fluid administration to, and/or withdrawal from, a vasculature of the live subject. In some embodiments, a vascular access port may include a single cavity or several distinct cavities, covered with one or several distinct septum members, provided as a single element or as several interconnectable members, some or all can be provided in the port body or in several portions or members of the vascular access port configured each as a separate port body. Before or after implantation, a catheter may be attached to the vascular access port with a distal end that physically enters the vasculature of the patient. Once connected, a lumen of the catheter is provided in direct fluid communication with the port body cavity.

A “vascular access port” as described herein, or a kit comprising it, may include or not include such a catheter and may include or not include a fitting for such a catheter. A vascular access port may have additional components and functionality not associated with fluid delivery or withdrawal. A vascular access port may be referred to herein as simply a “port” or an “implant”. A “subcutaneous port” refers to a vascular access port and optionally, more generally, to any other medical implantable port, configured particularly for implantation beneath skin tissues and is accessible by way of needle puncture or penetration thereinside, percutaneously, through skin tissues covering it.

The port body may be structurally and/or functionally configured for facilitating at least the basic function of the vascular access port of repeated accumulating and/or delivering and/or withdrawing fluid to or from subject's vasculature, and it may optionally lack or be initially configured without one or more other features, optional or vital ones, for facilitating additional functions associated with delivery, deployment and/or prolonged use of a vascular access port. The port body may be connected to at least one other component for providing the vascular access port additional features or capabilities, for example improved or easier deliverability, selective fixation to body tissues surrounding the port body and/or increased stability in a chosen implantation site such as a preformed subcutaneous void.

Deploying the vascular access port includes at least inserting the port body into a target implantation site in the subject body, such that a superior portion of the port body is accessible to repeated fluid transfer access. Vascular access port deployment may include compacting of tissue mass surrounding periphery of the port body thereby increasing a volume of a void formed in the target implantation site between the periphery of the port body and the compacted tissue mass. The void can be a subcutaneous void located between or beneath skin tissue layers at the target implantation site. Concurrently with increasing the void volume, or immediately afterwards, the increased void volume is occupied with the vascular access port such as by increasing the volume of the port body or by connecting one or more solid shaped components (e.g., a port body extension) thereto. This also includes the situation that the tissue mass compaction may be a direct result of such increase in port body volume. The compacted tissue mass normally affects a continuous pressure on the deployed vascular access port and thereby increase its fixation and/or stability in the subcutaneous void. The port body may include an inferior portion which defines a cavity and a superior portion coupled with a septum member covering the cavity, and the vascular access port may be deployed such that the compacted tissue mass surrounds only an inferior portion and not the superior portion of the port body.

A method for implanting a subcutaneous port may include at least one of the followings (not necessarily in same order):
forming a surgical opening across skin layers of a subject, the surgical opening comprising an opening neck portion enclosing and restricting a maximal opening circumference;
creating a subcutaneous void beneath the skin layers via the surgical opening;
pushing the subcutaneous port into the subcutaneous void via the surgical opening, the subcutaneous port has a predetermined spatial shape having a maximal cross sectional circumference greater than the maximal opening circumference when in an elastically relaxed state, the subcutaneous port is locally elastically compressible along a length thereof. Such pushing is configured to force each segment of the subcutaneous port pressing against the opening neck portion to maintain or reduce to a segment circumference being equal to or smaller than the maximal opening circumference, thereby allowing squeezing of the subcutaneous port through the surgical opening.

Implantation of the subcutaneous port is optionally combined in implantation of a catheter, such that one end of the catheter is connected to the port and maintain fluid communication with its cavity while the other end of the catheter is positioned in the patient's vasculature, optionally in the superior vena cava or in the right atrium, Therefore, the method may further comprise at least one of the followings (not necessarily in same order):
accessing into the jugular vein with a needle
inserting a wire into the jugular vein through the needle
removing the needle from the jugular vein,
inserting a dilator into the jugular vein over the wire,
inserting a peel apart sheath into the jugular vein over the wire,
removing the wire and/or the dilator from the jugular vein,
inserting a catheter into the jugular vein through the peel apart sheath, and
removing the peel apart sheath from the jugular vein,

The method may further include at least one of the followings (not necessarily in same order):
advancing the catheter via the jugular vein to the superior vena cava,
confirming under imaging position of a distal tip of the catheter in the superior vena cava or in the right atrium, and
adjusting the position of the catheter distal tip.

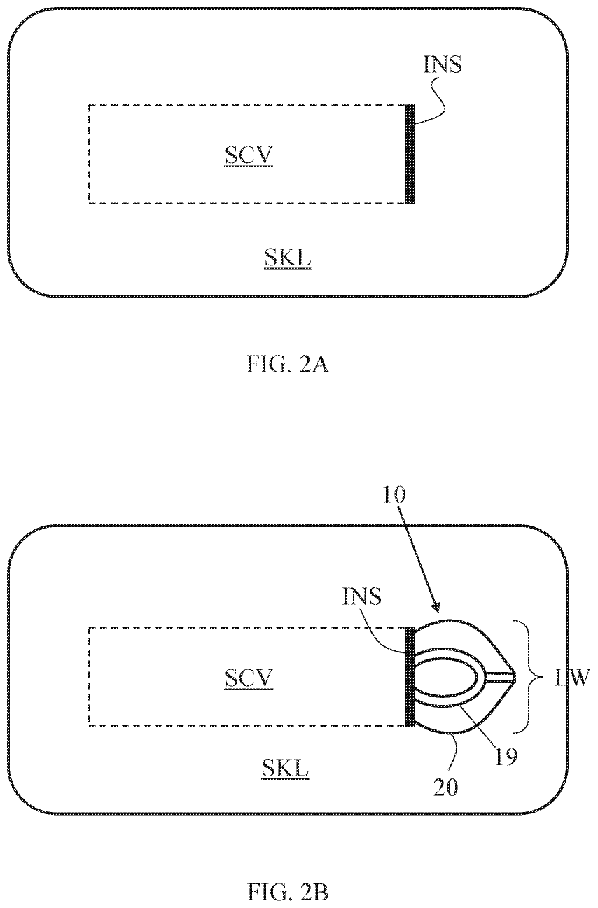

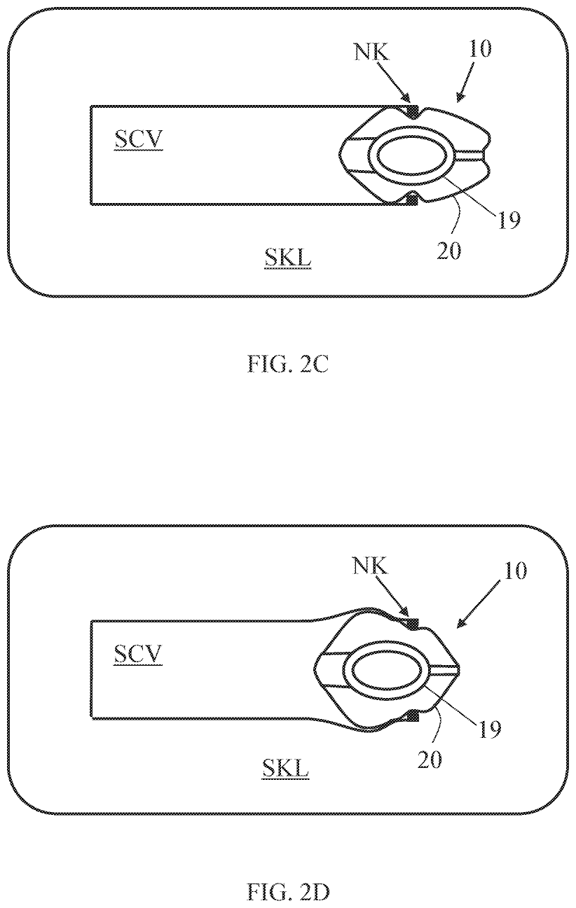

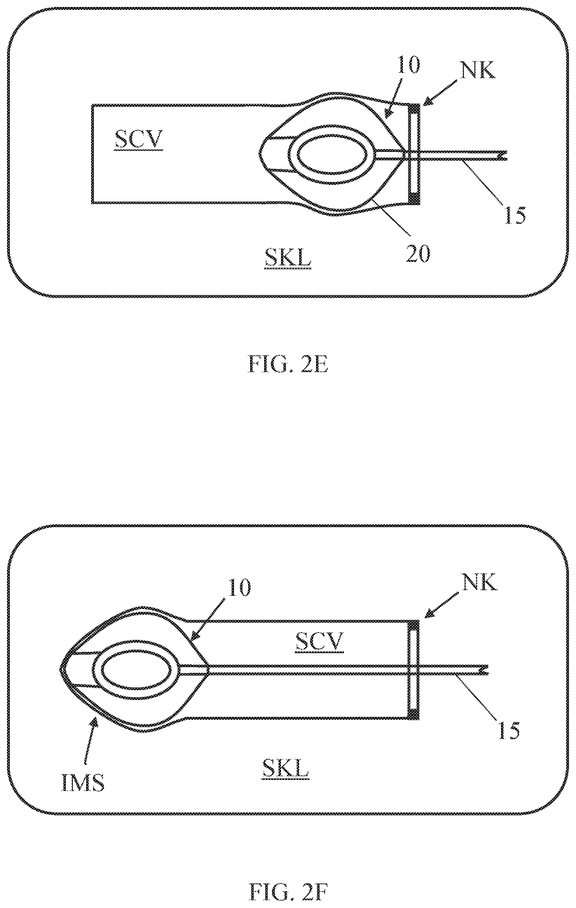

FIGS. 2A-2F schematically illustrate exemplary scenarios representing steps in an exemplary procedure for implanting squeezable subcutaneous port 10 in a body of a subject (patient). As shown in FIG. 2A, a surgical opening, optionally in a form of a cut or incision INS, is first made through subject's skin layers SKL, such as by using a scalpel; then, a subcutaneous pocket, tunnel and/or void. SCV can be formed such as by using a Kelly clamps, a needle holder (such as needle holder 119 shown in FIG. 6) or any other appropriate instrument. Size (e.g., width, circumference and/or length) of incision INS can be determined according to size of port 10 in use, the medical need and duration, patient's condition, and/or patient's body and anatomical consideration (including skin condition, weight), gender considerations, or others. Size of incision INS can be taken as a portion of port's 10 largest width LW, for example about 95% or less, about 75% or less, or about 50% or less; and/or smaller than port's 10 largest width LW by at least 1 mm, by at least 5 mm, or by at least 10 mm, for example.

Port 10 can then be forcefully pushed through incision INS into subcutaneous void SCV with sufficient force to deform it elastically such that it can be squeezed through opening neck portion NK, formed in skin layers SKL by incision INS, until port 10 can elastically regain an elastically less-stressed or non-stressed form within subcutaneous void SCL, as shown in FIGS. 2B to 2F, Such squeezing of port 10 through opening neck portion NK is facilitated by lateral compression against inner member 19, locally and gradually along length of port 10, of the portion of outer member 20 being in direct contact with opening neck portion NK. An exemplary sequence of such ‘squeezing in’ of port 10 is shown in FIG. 2C and FIG. 2D. Outer member 20 is configured such that it can expand elastically once it is partially or completely provided in subcutaneous void SCV distally to opening neck portion NK, optionally by pushing or compacting surrounding soft tissues normally located beneath skin layers SKL, as shown in FIG. 2E. Port 10 can be pushed further distally until reaching target implantation site IMS, where it can be optionally fixated or left without additional fixation to surrounding tissues, as shown in FIG. 2F. Before, during or after implantation sequence and/or squeezing in of port 10, it can be connected to catheter 15.

In similar considerations, the squeezable subcutaneous port can be found applicable for removing thereof through smaller surgical openings. A method for removing a subcutaneous port from a body of a subject may include at least one of the followings (not necessarily in same order):
forming a surgical opening across skin layers of the subject, the surgical opening comprising an opening neck portion enclosing and restricting a maximal opening circumference;
creating a subcutaneous passage beneath the skin layers between the surgical opening and a proximal end of the subcutaneous port; and
pulling the subcutaneous port through the subcutaneous passage and the surgical opening for removing it from the patient, the subcutaneous port has a predetermined spatial shape having a maximal cross-sectional circumference greater than the maximal opening circumference when in an elastically relaxed state, the subcutaneous port is locally elastically compressible along a length thereof. Such pulling is configured to force each segment of the subcutaneous port pressing against the opening neck portion to maintain or reduce to a segment circumference being equal to or smaller than the maximal opening circumference, thereby allowing squeezing of the subcutaneous port through the surgical opening.

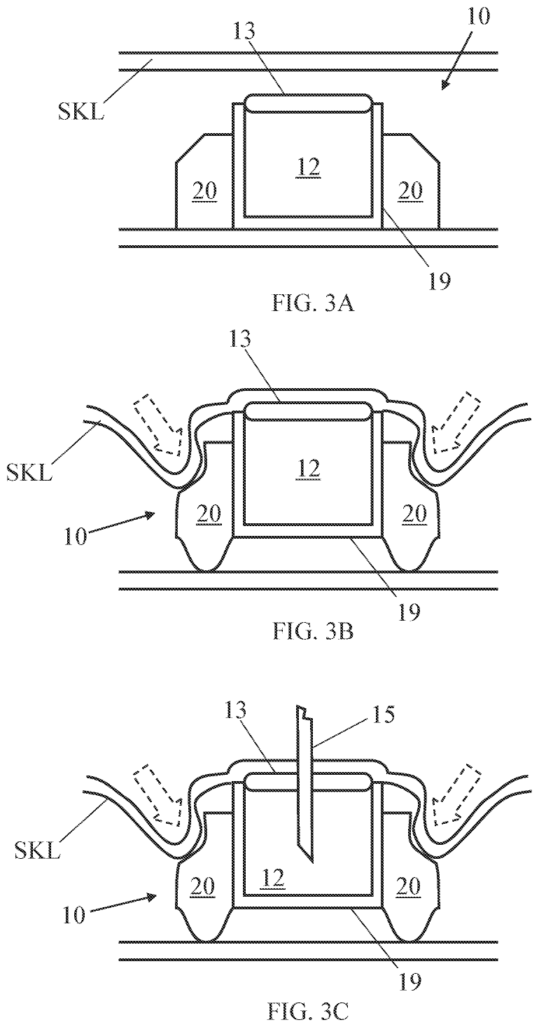

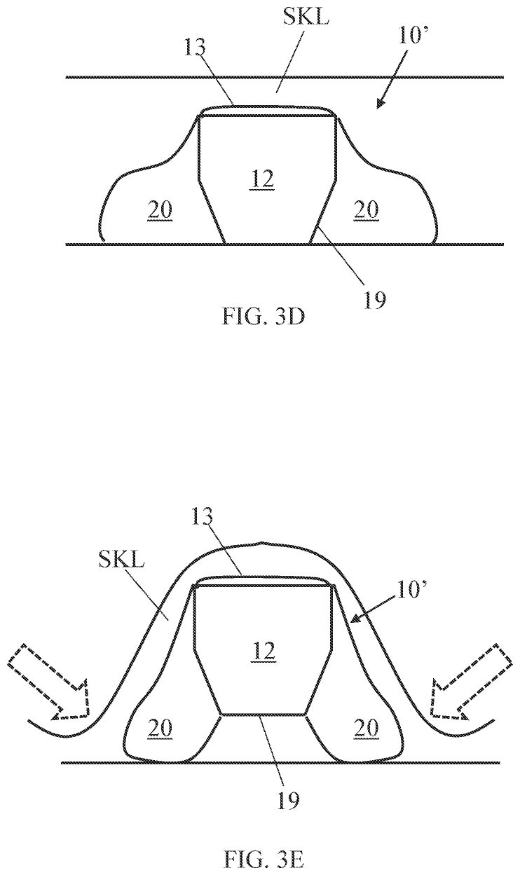

FIGS. 3A-3C schematically illustrate exemplary scenarios representing steps in an exemplary procedure for accessing and operating port 10 (shown in frontal cut views), already implanted and provided in subcutaneous void SCV. As shown in FIG. 3A in regular state, port 10 may not reach or press with its superior surface or with septum member 13 skin layers SKL in a manner that forms any visible or tactile protrusion or bulging for allowing medical practitioner easier location and access to port 10. Advantages for this ‘hidden mode’ in regular state is reduction or prevention of infections and wounding of surrounding tissues, as well as patient's aesthetical or emotional related preferences. Alternatively, port 10 may be sized and configured so as to cause constant visible or tactile protrusion or bulging through the overlaying skin. Additionally or alternatively, port 10 may have special purpose bulging portions or mechanical, electronical or other means to facilitate or ease in locating thereof, optionally selectively or automatically when needed. As shown in FIG. 3B, the medical practitioner can apply (e.g., with her fingers) compression forces through the overlaying skin, directly to outer member 20, so as to deform it elastically such that it increases in height and can therefore cause elevation of port 10 to sufficiently cause local skin protrusion and ease in locating of the septum member 13 beneath the skin. Once port 10 is in a chosen height and/or the septum member 13 is properly located, the medical practitioner can access into cavity with needle 14 through skin layers SKI and septum member 13, as shown in FIG. 3C.

Another elevating technique may be applied with port 10 or with a variation 10′ thereof configured with flexible or bendable (in a superior-to-inferior direction). FIG. 3D shows port 10′ in a regular state similarly to as shown in FIG. 3A, whereby it may not reach or press with its superior surface or with septum member 13 skin layers SKL in a manner that forms any visible or tactile protrusion or bulging for allowing medical practitioner easier location and access to port 10′, FIG. 3E illustrates a second exemplary scenario wherein outer member 20 is pressed via skin layers SKL, from both sides of inner member 19, thereby elastically flexing or bending outer member 20 such that lateral-peripheral portions thereof shift inferiorly away (below base of) and laterally towards inner member 19. This increases in height and can therefore cause elevation of port 10′ to sufficiently cause local skin protrusion and ease in locating of the septum member 13 beneath the skin. Once port 10′ is in a chosen height and/or the septum member 13 is properly located, the medical practitioner can access into cavity 12 with needle 14 through skin layers SKL and septum member 13, similarly to as shown in FIG. 3C.

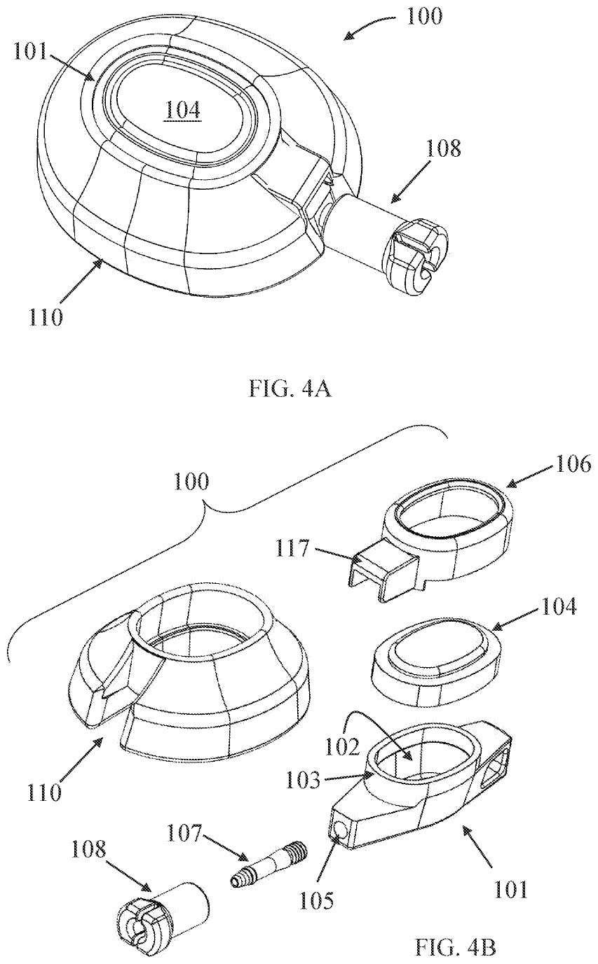

FIGS. 4A-4B respectively illustrate an exemplary squeezable subcutaneous port 100 in an assembled isometric view and in an exploded isometric view. FIGS. 5A-5B respectively illustrate port 100 in a side cross-sectional view and in a frontal cross-sectional view. Port 100 is optionally an exemplary embodiment, representation, or variation of port 10, and may include some or all structural and/or functional features described with respect to port 10. Port 100 in an elastically relaxed state may have a maximal width of 50 mm or less, optionally 25 mm or less; a maximal height of 30 mm or less, optionally 15 mm or less; and a maximal length (with or without catheter connecting means) of 50 mm or less, optionally 30 mm or less. In some embodiments, port 100 is configured for squeezing through surgical openings (without further widening or tearing when passing therethrough) having a maximal opening circumference of about 80 mm or less, optionally of about 60 mm or less, optionally of about 40 mm or less, and/or formed by a surgical incision of about 20 mm or less in length, optionally about 15 mm or less in length, or optionally about 10 mm or less in length.

Port 100 includes a rigid inner member 101 comprising a cavity 102 opened to a first cavity opening 103 and to a second cavity opening 105. First cavity opening 103 is closed with a septum member 104 and configured for repeated needle penetrations therethrough into cavity 102. Second cavity opening 105 is configured for facilitating fluid communication between cavity 102 and a lumen of a catheter, Inner member 101 is configured with sufficient rigidity to accommodate (safely and efficiently) a chosen length of a needle and to prevent the needle's tip from penetrating therethrough. Septum member 104 is optionally oval, as shown, although it may have any other shape.

A cap member 106 is coupled over septum member 104 and over the superior portion of inner member 101 to form a unitary rigid encapsulated core body of port 100. Septum member 104 is restrained in-position and optionally compressed, at least partly, by and in-between cap member 106 and inner member 101. Inner member 101 and/or cap member 106 are optionally formed of hard plastic such as PEEK, or from metal such as titanium or stainless-steel alloys. Cap member 106 is optionally fixedly connected to inner member 101, such as by way of adhesives, compressing fitting and/or welding (e.g., ultrasonic welding if the parts are made of plastic, or laser welding if the parts are made of metal). The encapsulated core body, once fully assembled, has sufficient rigidity and yield strength, and is configured to maintain internal pressures that are common during injections into cavity 102 (of optionally about 5 ml/sec injections at 300 psi, or higher or lower). A lumen extension 107 is coupled to inner member 101 with distal portion thereof extending towards cavity 102 through second cavity opening 105 and configured to provide a fluid-tight passage via proximal portion thereof to a catheter lumen. A connector member 108 is coupled over lumen extension 107 and is configured to facilitate selective connection of a catheter distal end with port 100, such as with a luer-fitting based connection mechanism.

Port 100 includes a rigid grasping portion 117 provided at proximal end thereof and is configured for facilitating efficient and safe grasping of port 100 with grasping means, such as Kelly clamps or surgical needle holder. Rigid grasping portion 117 may be provided as a proximal extension of cap member 106, as shown, and located above (superiorly to) lumen extension 107 and connector member 108. FIG. 6 illustrates port 100 grasped at rigid grasping portion 117 with an exemplary surgical needle holder 119. Rigid grasping portion 117 is shown with its flat surface extending horizontally so that needle holder 119 can be held by the medical practitioner having its arms arranged vertically (one over the other), as shown in FIG. 6. Alternatively, rigid grasping portion 117 can be arranged with its flat surface in any other direction, including optionally vertically. Rigid grasping portion 117 is optionally configured in size, surface area of its flat surface, thickness and/or durability and/or strength to facilitate firm grasping by needle holder 150 sufficiently to push, squeeze-in by elastic compression, and maneuver port 100 through a surgical opening smaller than its maximal relaxed dimensions, without releasing grip or mechanical failure. Needle holder 150 can be used to form or increase size of a subcutaneous void before grasping on to port 100 and delivering it into the subcutaneous void.

In some embodiments, inner member 101 can be functionally configured or applicable to serve as a vascular access port although it may be incapable, insufficient, or less compatible of providing one or more, optionally essential, features for improving, facilitating or easing implantation and/or long-term use of port 100. Port 100 includes an outer member 110 comprising of flexible material and configured for providing one or more additional features, including but not limited to: stability and/or fixation in implantation site, transdermal accessibility, identification and/or locating of septum member 104 for repeated percutaneous fluid administration, protection to port body and/or overlaying skin layers, or others.

In some embodiments, subcutaneous port 100 is configured to squeeze into a subcutaneous void when pushed through a surgical opening greater than a maximal cross-sectional circumference of inner member 101 and smaller than a maximal cross-sectional circumference of port's 100 predetermined spatial shape in an elastically relaxed state. In order to facilitate such ‘squeezing’ property through narrow surgical openings, the elastic, optionally soft and pliable, outer member 110 adds significant width, cross section and/or volume to inner member 101, at least around lateral (sides) periphery thereof, optionally particularly around lower (inferior) portions thereof, thereby providing sufficient material and space to compress at normal forces commonly applied for introducing ports or other implants through surgical or other opening into a body of a live subject. In some such embodiments, port 100 is greater than inner member 101 alone by at least 25%, optionally by at least 50%, optionally by at least 75%, optionally by at least 100%, in width, in area and/or in volume, in a maximal axial cross section of the predetermined spatial shape (shown in FIG. 5B, for example). As used herein, the term “axial cross section” refers to a cross section of an object (e.g., port 100, for example) provided along a plane (e.g., horizontal plane) that intersects a longitudinal axis of the object at a right angle (e.g., transversely). The term “maximal axial cross section” refers herein to a particular axial cross section of the object being greatest (or equal to) in total area than all other axial cross sections of the object,

Outer member 110 is locally elastically compressible laterally towards the lateral periphery portion of inner member 101, and port 100 is thus configured to reduce in maximal width by at least 10% when outer member 110 is compressed under a force greater than 5 N, and/or by at least 25% when outer member 110 is compressed under a force greater than 20 N. In some embodiments, a subcutaneous port configured as port 100 and having maximal width of about 21.7 mm (in maximal axial cross section thereof), was found to compress by about 1.5 mm (about 7%) under a normal force of about 0.45 N (Newton), by about 4.5 mm (about 21%) under normal force of about 3 N, by about 7.5 mm (about 34.5%) under normal force of about 8 N, and by about 10.5 mm (about 48%) under normal force of about 33 N.

Outer member 110 is connected to inner member 101 along at least one lateral periphery portion thereof, thereby forming a chosen predetermined spatial shape of the subcutaneous port when in an elastically relaxed state. Optionally, outer member is configured as a skirt or ring-like element encompassing most or all periphery of inner member 101, and optionally also periphery of cap member 106, in at least a circumferential segment thereof. In order to maintain sufficient rigid pushability of port 100 for its insertion and implantation, the rigid inner member 101 extends longitudinally along most or all length of port 100, to function also as a rigid spine-like structure of port 100, optionally in combination with cap member 106. Inner member 101 includes a distal (front) portion 113 extending distally relative to 102 cavity, having a rounded or pointed leading edge 116 configured to facilitate or ease penetration of port 100 via the surgical opening. Port 100 may be configured such that distal portion 113 is uncovered by outer member 20 which may extend distally and transversely therefrom, although (as shown) it may be covered with a thin layer of outer member 110 such that sufficient rigid pushability is substantially uncompromised.

Outer member 110 varies in width and/or thickness in at least one direction relative to inner member 101, and may be greater in width and/or thickness along an intermediate portion 111 and/or along a proximal portion 112 of inner member 101 than along distal (front) portion 113 of inner member 101. For example, width and/or thickness of outer member 110 along intermediate portion 111 may be about 3 mm or more, optionally about 5 mm or more, in each side of inner member 101, and about 2 mm or less, or optionally about 1 mm or less, along distal portion 113 of inner member 101. Similarly, outer member 110 may be greater in width and/or thickness along an inferior portion 114 than along a superior portion 115 of inner member 101. For example, width and/or thickness of outer member 110 along inferior portion 114 may be about 5 mm or more, optionally about 7 mm or more, and may be about 3 mm or less, or optionally about 2 mm or less, along superior portion 115, in each side of inner member 101.

Outer member 110 is optionally made of silicone or other flexible and elastic polymer or rubber, and is optionally extruded, casted or molded over periphery of inner member 101 or over periphery of the encapsulated core body (i.e., the structure formed by the interconnected inner member 101, septum member 104 and cap member 106), optionally within boundary of a chosen shaped mold, when forming subcutaneous port 100. Outer member 110 is configured with hardness equal to or smaller than about 50 Shore A, optionally equal to or smaller than about 35 Shore A, optionally equal to or smaller than about 20 Shore A, or optionally equal to or smaller than about 00-50 Shore.

Outer member 110 is configured with elastic resistance to compression or compressive strength sufficient to maintain the predetermined spatial shape within a surgically formable subcutaneous void (such as subcutaneous void SCV described above) when under naturally occurring subcutaneous stresses. In some embodiments, outer member 110 has compressive strength equal to or smaller than about 50 MPa, optionally equal to or smaller than about 35 MPa, optionally equal to or smaller than about 20 r″, 1 Pa., or optionally equal to or smaller than about 10 MPa.

Similarly to outer member 20, outer member 110 is locally compressible against inner member 101 and configured to substantially maintain a constant overall volume by enlarging remotely to a compressed region thereof. As such, outer member 110 is configured to facilitate squeezing of port 100 into the subcutaneous void when pushed through a surgical opening greater than a maximal cross sectional circumference of inner member and smaller than a maximal cross sectional circumference of the predetermined spatial shape. In some embodiments, outer member 110 is locally compressible against inner member 101, at least in its inferior portion, to about 50% or less, optionally to about 25% or less, its elastically unstressed width, in each side of inner member 101. Maximal compression is optionally adjacent to the maximal cross-sectional circumference of the predetermined spatial shape. Sufficient compression for squeezing through narrow surgical openings, as described, is optionally reached when port 100 is forced through the surgical opening with an axial (pushing) force of about 10 kgf or less, optionally about 5 kgf or less, optionally about 3 kgf or less, optionally about 2 kgf or less, or optionally about 1 kgf or less.

In some embodiments, outer member 110 is configured to lengthen when it is locally compressed radially against inner member 101, thereby increasing overall length of port 100. Outer member 110 is configured to lengthen mostly or only proximally and optionally inner member 101 is configured to resist or prevent the outer member 110 from lengthening distally. Furthermore, outer member 110 is configured to expand perpendicularly to the compression axis when locally compressed against inner member 101, thereby increasing overall height of port 100. Outer member 110 is configured to expand mostly or only inferiorly and optionally inner member 101 is configured to resist or prevent outer member 110 from expanding posteriorly.

As shown in FIG. 5B, outer member 110 is formed as a single component optionally as a solid member occupying substantially most or all volume thereof around inner member 101. FIG. 7A illustrates port 100 with another exemplary outer member 120 (shown separately in FIGS. 7B and 7C), which includes an (empty) elastic shell-like structure 121. Structure 121 comprises a thin layer 122 enclosing an outer member cavity 123 around inner member 101. Thin layer 122 may be 0.5 to 2 mm thick, and outer member cavity 123 may occupy at least 50%, optionally at least 75%, or optionally at least 90%, of port 100 volume around inner member 101. FIGS. 5A-8C illustrate an exemplary variant 120′ of outer member 120, which includes elastic shell-like structure 121 now filled with a flexible filler 124. Shell-like structure 121 is formed of a first material and filler 124 is formed of a second material, wherein the first material differs from the second material by at least one of rigidity, elasticity and compression strength. The hardness of the first material is optionally about 30 Shore A to about 50 Shore A, and the hardness of the second material is optionally about 00-20 Shore to about 20 Shore A.

FIG. 9A illustrates port 100 with another exemplary outer member 125, shown separately in FIGS. 9B and 9C, which includes a plurality of thru holes 126. Thru holes 126 optionally occupying at least 20%, optionally at least 50%, of the outer member 125 total volume. FIG. 10A illustrates port 100 an outer member 130 of a different type, shown separately in FIGS. 10B and IOC. Outer member 130 includes a plurality of thin rib-like members 131 extending perpendicularly from each side of inner member 101. Rib-like 131 members are spaced apart from each other sufficiently to allow a first phase of partial flexing of the rib-like members without contacting each other, when port 100 is pushed through the surgical opening. This allows a different type of squeezing mechanism, by which distinct flexing or bending of one or few rib-like members 131 replaces local radial compression of outer member body. FIG. 11A illustrates port 100 with a different outer member 135, shown separately in FIG. 11B, having a base footprint 136 shape formed of sequentially joined oval segments 137 forming narrowed neck portions 138 therebetween, unlike the substantially (single) oval shaped base footprint of outer member 110, for example. This can allow passage of port 100 via a smaller surgical opening using a series of squeezing episodes per each oval segment 137, rather than a single squeezing effort of the entire outer member.

FIGS. 12A-12D schematically illustrate exemplary scenarios representing steps in an exemplary procedure for implanting an exemplary subcutaneous port 150 in a subcutaneous void SCV by pushing (e.g., squeezing) it through a surgical opening like incision INS Port 150 may be similar or identical in some or all structural and/or functional features of port 10, and includes flexible outer member 20 connected to rigid inner member 19 along at least one lateral periphery portion thereof and forming a chosen predetermined spatial shape for port 10 when in an elastically relaxed state. Surgical opening INS is greater than a maximal cross-sectional circumference of inner member 19 and smaller than a maximal cross-sectional circumference of the predetermined spatial shape of port 150. Unlike port 10, port 150 also includes elastic extensions 151 projecting from inner member 19, embedded in the flexible material forming outer member 20 and surrounding the at least one lateral periphery portion of the inner member 19. In some embodiments, elastic extension 151 are configured to increase resistance to lateral compression and/or to facilitate or increase distribution of loads around periphery of inner member 19 originating at localized lateral compression of outer member 20 towards elastic extension 151, relative to mechanical properties of the flexible, and optionally soft and pliable, material constructing outer member 20.

FIG. 12A illustrates port 150 in an elastically relaxed state with no external stresses applied thereto or ones able to compress portions of outer member 20 and/or to press elastic extensions 151 laterally towards inner member 19, for example. Ms. 12B and 12C illustrate, respectively, an initial stage and an advanced stage of port 150 squeezing through surgical opening IN As shown, portion of outer member 20 in contact with, and pressed against, adjacent skin portion surrounding surgical opening INS is locally pressed and causes elastic extensions 151 to shift laterally inwardly and proximally relative to inner member 19, Since that elastic extension 151 are embedded in outer member 20, their motion and new position directly affects deformation of portions of outer member 20, particularly portions located proximally to surgical opening INS.

In some embodiments and as shown, such deformation of outer member 20, caused by elastic extensions 151 shifting towards inner member 19, results in compression (e.g., reduction in width) along most or all length outer member 20 at least proximally to surgical opening INS, and/or to extending (e.g., increase in length) of outer member 20 at least in a proximal direction (e.g., in a direction pointing from distal to proximal). In some embodiments, overall volume of outer member 20 is substantially maintained while port 150 is squeezed through surgical opening INS, or is reduced by about 15% or less, or optionally by about 10% or less, or optionally by about 5% or less. FIG. 12D shows port 150 after it has been fully implanted in subcutaneous void SCV. In some embodiments and as shown, subcutaneous implant 150 substantially resumes to its predetermined spatial shape, optionally because subcutaneous void SCV is sufficiently large and/or contracting force applied on outer member by bodily tissues surrounding or forming subcutaneous void SCV are too small for substantially shifting elastic extensions 151 and/or compressing outer member 20.

FIG. 13 illustrates an exemplary vascular access port system 160 comprising an exemplary subcutaneous port 200 connected to a catheter 170. FIGS. 14A-14I illustrate several views of subcutaneous port 200. Port 200 is optionally an exemplary configuration, representation, or variation of port 150, and may include some or all structural and/or functional features described with respect to port 150. Subcutaneous port 200 includes a rigid inner member 201 and an outer member 210 comprising of a flexible and optionally soft and pliable material. FIGS. 14A and 14B show top views of port 200 with solid and transparent representation of outer member 210, respectively. FIG. 14C shows a maximal axial cross section of port 200. FIGS. 14D and 14E show front-top view angle isometric projections of port 200 with and without outer member 210, respectively, FIGS. 14F and 14G show rear-bottom view angle isometric projections of port 200 with and without (transparent) outer member 210, respectively. FIGS. 14H and 14I show isometric views of port 200 absent of outer member 210, with and without a cap member.

Subcutaneous port 200 has a predetermined (e.g., preformed) spatial shape, when its members are in an elastically relaxed state. When in the elastically relaxed state, port 200 may have a maximal width of 50 mm or less, optionally 25 mm or less; a maximal height of 30 mm or less, optionally 15 mm or less; and a maximal length (with or without catheter connecting means) of 50 mm or less, optionally 30 mm or less. In some embodiments, port 200 is configured for squeezing through surgical openings (without further widening or tearing when passing therethrough) having a maximal opening circumference of about 80 mm or less, optionally of about 60 mm or less, optionally of about 40 mm or less, and/or formed by a surgical incision of about 20 mm or less in length, optionally about 15 mm or less in length, or optionally about 10 mm or less in length.

Inner member 201 includes (encloses) a cavity 202 opened to a first cavity opening 203 closed with a septum member 204 configured for repeated needle penetrations therethrough into cavity 202. A cap member 206 is coupled over septum member 204 to a superior portion of inner member 201 to form a unitary rigid encapsulated core body of port 200, configured to withstand power injection pressures generatable within cavity 202. Cavity 202 is also opened to a second cavity opening 205 configured for facilitating fluid communication between cavity 202 and a lumen of catheter 170. Inner member 201 includes a distal portion 209 extending distally relative to cavity 202, having a rounded or pointed leading edge configured to facilitate or ease penetration of port 200 via a narrow surgical opening. Inner member 201 includes a rigid grasping portion 208 at proximal end 207 thereof configured for facilitating grasping with grasping means, such as Kelly clamps or surgical needle holder.

Outer member 210 is connected to inner member 201 along lateral periphery portion 211 thereof and forms a predetermined spatial shape of port 200 (as shown), when in an elastically relaxed state. Subcutaneous port 200 is configured to squeeze into a subcutaneous void when pushed through a narrow surgical opening indicative as being greater than a maximal cross-sectional circumference of inner member 201 and smaller (e.g., by at least 10%, or by at least 25%) than a maximal cross-sectional circumference of the predetermined spatial shape.

Subcutaneous port 200 also includes an elastic extension 212 in a form of a thin rib-like or ring-like element encircling (most or all) lateral periphery 211 of the inner portion 201, and is fixed to inner member distal portion 209 and projecting proximally and laterally—outwardly therefrom to surround lateral periphery portion 211 of inner member 201. Extension 212 is embedded in the flexible material forming outer member 210, and is substantially stiffer and has greater resistance to flexing or bending than the flexible material, and forms a gap 214 with lateral periphery portion 211 which is filled with the flexible material. As such, extension 212 is configured to distribute compressing loads originating from a locally compressed portion of outer member 210 to other portions of the outer member 210. Elastic extension 212 is allowed to flex axially (e.g., proximally) and/or laterally (e.g., outwardly) relatively to inner member 201 with proximal portions thereof distant to inner member distal portion 209. When port 200 is squeezed through such a narrow surgical opening, outer member 210 is configured to compress elastically laterally towards lateral periphery portion 211 of inner member 201, and therefore to compress laterally and/or extend proximally most or all outer member 210. Outer member 210 is locally compressible against inner member 201 sufficiently for squeezing through a narrow surgical opening when the subcutaneous port 200 is pushed with normal manual forces, such as an axial force equal to or smaller than about 5 kgf.

In some embodiments, subcutaneous port 200 is configured to squeeze into a subcutaneous void when pushed through a surgical opening greater than a maximal cross-sectional circumference of inner member 201 and smaller than a maximal cross-sectional circumference of port's 200 predetermined spatial shape in an elastically relaxed state. In order to facilitate such ‘squeezing’ property through narrow surgical openings, the elastic, optionally soft and pliable, outer member 210 adds significant width, cross section and/or volume to inner member 201, at least around lateral (sides) periphery thereof, optionally particularly around lower (inferior) portions thereof, thereby providing sufficient material and space to compress at normal forces commonly applied for introducing ports or other implants through surgical or other opening into a body of a live subject. In some such embodiments, port 200 is greater than inner member 201 alone by at least 25%, optionally by at least 50%, optionally by at least 75%, optionally by at least 100%, in width, in area and/or in volume, in a maximal axial cross section of the predetermined spatial shape (shown in FIG. for example). Outer member 210 is locally elastically compressible laterally towards the lateral periphery portion of inner member 201, and port 200 is thus configured to reduce in maximal width by at least 10% when outer member 110 is compressed under a force greater than 5 N, and/or by at least 25% when outer member 110 is compressed under a force greater than 20 N. In some embodiments, a subcutaneous port configured as port 200 and having maximal width of about 21.7 mm (in maximal axial cross section thereof), was found to compress by about 1.5 mm (about 7%) under a normal force of about 1.7 N (Newton), by about 4.5 mm (about 21%) under normal force of about 11 N, by about 7.5 mm (about 34.5%) under normal force of about 30 N, and by about 9 mm (about 41%) under normal force of about 50 N.

Outer member 210 includes or is filled with one or more materials including the flexible material, or have portions differentiating in one or more mechanical properties such as hardness. Nevertheless, outer member 210 retains overall flexibility and/or softness sufficient to allow squeezability as described. The flexible material forming outer member 210 includes and is formed of soft elastomer and/or silicone rubber configured with hardness equal to or smaller than about 50 Shore A, optionally equal to or smaller than about 35 Shore A, optionally equal to or smaller than about 20 Shore A, or optionally equal to or smaller than about 00-50 Shore. In some embodiments, the flexible material fills most or all space formed in the predetermined spatial shape around inner member 201, although one or more voids or holes may be distributed in outer member 210. Such voids may be filled with a different material or with same materials configured with different one or more mechanical characteristics. In some embodiments, outer member 210 is formed as a single component, optionally by way of extruding, casting or molding the flexible material over inner member 201 and extension 212 within restricting spatial boundaries shaped according to the predetermined spatial shape.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’. ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’. ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), features), charactetistic(s), parameter(s), integer(s), step(s), or groups thereof. Each of these terms is considered equivalent in meaning to the phrase ‘consisting essentially of’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about ° C. and about 25° C.’, and is considered equivalent to, and meaning the same as, the phrase ‘room temperature refers to a temperature in a range of from about 20° C. to about 25° C.’.

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

All publications, patents, and or/and patent applications, cited or referred to in this disclosure are herein incorporated in their entirety by reference into the specification, to the same extent as if each individual publication, patent, or/and patent application, was specifically and individually indicated to be incorporated herein by reference in addition, citation or identification of any reference in this specification shall not be construed or understood as an admission that such reference represents or corresponds to prior art of the present invention. To the extent that section headings are used, they should not be construed as necessarily limiting.

1. A subcutaneous port, comprising:
a rigid inner member comprising a cavity opened to a first cavity opening closed with a septum member, configured for repeated needle penetrations therethrough into the cavity, and to a second cavity opening configured for facilitating fluid communication between the cavity and a lumen of a catheter; and
an outer member comprising of flexible material connected to the inner member along at least one lateral periphery portion of the inner member thereby forming a predetermined spatial shape of the subcutaneous port when in an elastically relaxed state;
wherein the subcutaneous port is configured to squeeze into a subcutaneous void when pushed through a surgical opening greater than a maximal cross-sectional circumference of the inner member and smaller than a maximal cross-sectional circumference of the predetermined spatial shape;
wherein the subcutaneous port is configured to reduce in maximal width by at least 10% when the outer member is compressed under a force greater than 5 N, and/or by at least 25% when the outer member is compressed under a force greater than 20 N.

2. The subcutaneous port according to claim 1, wherein the subcutaneous port in the elastically relaxed state is greater than the inner member by at least 50% in width, in area and/or in volume, in a maximal axial cross section of the predetermined spatial shape.

3-6. (canceled)

7. The subcutaneous port according to claim 1, wherein the flexible material includes soft elastomer and/or silicone rubber.

8. The subcutaneous port according to claim 7, wherein the flexible material fills most or all space formed in the predetermined spatial shape around the inner member.

9. The subcutaneous port according to claim 1, comprising at least one elastic extension stiffer than the flexible material, projecting from the inner member and surrounding the at least one lateral periphery portion of the inner member, wherein the at least one extension is embedded in the flexible material and configured to distribute compressing loads originating from a locally compressed portion to other portions of the outer member.

10. The subcutaneous port according to claim 9, wherein the at least one extension forms a gap with the at least one lateral periphery portion of the inner member filled with the flexible material.

11. The subcutaneous port according to claim 10, wherein the at least one extension is configured to approximate the at least one lateral periphery portion of the inner member when the outer member is compressed laterally and/or extended axially proximally relative to the inner member.

12. The subcutaneous port according to claim 9, wherein the at least one extension projects proximally and laterally-outwardly from a distal portion of the inner member located distally to the cavity.

13. The subcutaneous port according to claim 12, wherein the at least one extension is fixed to the inner member distal portion and allowed to flex axially and/or laterally relatively to the inner member with portions thereof distant to the inner member distal portion.

14. The subcutaneous port according to claim 9, wherein the at least one extension encircles or surrounds most or all lateral periphery of the inner portion.

15-24. (canceled)

25. A method comprising:
forming a surgical opening across skin layers of a subject, the surgical opening comprising an opening neck portion enclosing and restricting a maximal opening circumference;
creating a subcutaneous void beneath the skin layers via the surgical opening;
providing a subcutaneous port comprising a rigid inner member and a flexible outer member, and configured to reduce in maximal width by at least 10% when the outer member is compressed under a force greater than 5 N, and/or by at least 25% when the outer member is compressed under a force greater than 20 N;
pushing the subcutaneous port into the subcutaneous void via the surgical opening, the subcutaneous port has a predetermined spatial shape having a maximal cross-sectional circumference greater than the maximal opening circumference when in an elastically relaxed state, the subcutaneous port is locally elastically compressible along a length thereof;
wherein the pushing forces the subcutaneous port to elastically compress in diameter and/or extend proximally in length when pressed against the opening neck portion, thereby allowing squeezing of the subcutaneous port through the surgical opening.

26. The method according to claim 25, following the pushing, comprising allowing the subcutaneous port to voluntarily expand elastically up to the elastically relaxed state.

27. The method according to claim 25, wherein the creating and the pushing is performed with a surgical instrument.

28. The method according to claim 27, wherein the surgical instrument is a Kelly clamps or a surgical needle holder.

29. The method according to claim 25, wherein the subcutaneous port includes a flexible outer member connected to a rigid inner member along at least one lateral periphery portion of the inner member thereby forming the predetermined spatial shape of the subcutaneous port when in an elastically relaxed state.

30. The method according to claim 29, wherein the rigid inner member comprising a cavity opened to a first cavity opening closed with a septum member, configured for repeated needle penetrations therethrough into the cavity, and to a second cavity opening configured for facilitating fluid communication between the cavity and a lumen of a catheter.

31. The method according to claim 29, wherein the outer member is configured with elastic resistance to compression sufficient to maintain the predetermined spatial shape within a surgically formable subcutaneous void when under naturally occurring subcutaneous stresses.

32. The method according to claim 29, wherein the pushing forces the outer member to compress locally against the inner member while substantially maintaining an overall volume thereof by enlarging remotely to a compressed region thereof.

33. The method according to claim 25, further comprising at least one of: accessing into the jugular vein with a needle, inserting a wire into the jugular vein through the needle, removing the needle from the jugular vein, inserting a peel apart sheath and/or a dilator into the jugular vein over the wire, removing the wire and/or the dilator from the jugular vein, inserting a catheter into the jugular vein through the peel apart sheath, and removing the peel apart sheath from the jugular vein.

34. The method according to claim 33, further comprising at least one of: advancing the catheter via the jugular vein to the superior vena cava, confirming under imaging position of a distal tip of the catheter in the superior vena cava or in the right atrium, and adjusting the position of the catheter distal tip.
